(19) United States
(12) Patent Application Publication
(10) Pub. No.: US 2007/0134255 A1
(43) Pub. Date: Jun. 14, 2007

(54) Hmpv treatment with ribavirin and anti-hmpv antibody

(75) Inventors: Jeroen Maertzdorf, Utrecht (NL); James Henry Matthew Simon, Amsterdam (NL)

Correspondence Address: MORRISON & FOERSTER LLP, 12531 HIGH BLUFF DRIVE, SUITE 100, SAN DIEGO, CA, 92130-2040, US

(73) Assignee: ViroNovative B.V., Rotterdam (NL)

(21) Appl. No.: 10/555,329

(22) PCT Filed: May 3, 2004
(86) PCT No.: PCT/NL04/00293

§ 371 Date: May 15, 2006

(30) Foreign Application Priority Data
May 2, 2003 (EP) 03076299.1

(51) Int. Cl. A61K 39/42; A61K 31/7056

(52) U.S. Cl. 424/159.1; 514/43

(57) ABSTRACT

The invention relates to antimicrobial agents and antibodies and compositions comprising such agents and antibodies to treat and/or prevent respiratory and related diseases, in particular those caused by human metapneumovirus. Provided is a method for treating or preventing respiratory tract infections in a subject infected with a mammalian MPV, said method comprising administering a nucleoside analog, preferably Ribavirin or a derivative thereof, and an antimicrobial neutralising antibody, preferably an anti-hMPV antibody to said subject, and use of said nucleoside analog and antimicrobial neutralising antibody for the manufacture of a medicament for treating or preventing respiratory tract infections in a subject infected with a mammalian MPV.

The invention relates to the field of virology. More specifically, the invention relates to antimicrobial agents and antibodies and compositions comprising such agents and antibodies to treat and/or prevent respiratory and related diseases, in particular those caused by human metapneumovirus.

### INTRODUCTION

Human metapneumovirus (hMPV) is a respiratory viral pathogen that causes a spectrum of illnesses, ranging from asymptomatic infection to severe bronchiolitis. In 2001, van den Hoogen et al. (Nature Medicine 7, 719) described the identification of this new human viral pathogen from respiratory samples submitted for viral culture during the winter season. hMPV is a negative-sense nonsegmented RNA virus that has been categorized in the pneumovirus subfamily, family Paramyxoviridae, based on genomic sequence and gene constellation.

Little is known about the pathophysiology of hMPV infection, but hMPV appears to have a tropism for the respiratory epithelium. The respiratory viral pathogen hMPV causes a spectrum of illnesses, which ranges from asymptomatic infection to severe bronchiolitis. Paramyxoviridae, such as RSV, parainfluenza virus type 1, hMPV, and human parainfluenza virus type 3 (PIV3) are all known to cause clinical bronchiolitis. No medicine for the prevention or treatment of infectious disease caused by MPV is available thus far.

Many viruses have evolved their own specific enzymatic mechanisms to preferentially replicate virus nucleic acids at the expense of cellular molecules. There is often sufficient specificity in virus polymerases to provide a target for a specific antiviral agent, and this method has produced the majority of the specific antiviral drugs, RNA mutagens, currently in use. The majority of these drugs function as polymerase substrate (i.e. nucleoside/nucleotide) analogs, for example ribavirin. The mutagenicity of ribavirin results from the incorporation of ribavirin triphosphate opposite both cytidine and uridine in viral RNA.

Nucleoside analogs are (synthetic) chemical compounds created by modifying nucleosides, which are the natural building blocks of human and viral DNA and RNA. A nucleoside typically comprises a heterocyclic nitrogenous base, particularly a purine or pyrimidine, in N-glycosidic linkage with a sugar, particularly a pentose. Much antiviral and anticancer research has focused on nucleoside analogs, because viruses and cells use nucleosides to multiply. By mimicking the role of nucleosides in the cell division process, nucleoside analogs have been used to treat viruses and cancers by modifying the natural structure of DNA and RNA in a way that disrupts the viral and cellular replication machinery. Some nucleoside analogs have also been found to stimulate an antiviral immune response.

Ribavirin is currently indicated as an antiviral drug for the treatment of chronic hepatitis C. Also, the antiviral activity of Ribavirin was tested against RSV in vitro (Hruska 1980 Antimicrob. Agents Chemother. 17, 770) and in vivo (Hruska 1982 Antimicrob. Agents Chemother. 21, 125). Ribavirin has been used in a number of clinical trials to investigate its effectiveness in treating RSV infections, predominantly in infants and children with RSV infection and lower respiratory tract infection. Antiviral treatment with Ribavirin is currently the only approved antiviral prescription therapy for RSV. However, randomized trials comparing Ribavirin with placebo showed that Ribavirin does not always have positive effects.

Our finding that Ribavirin decreases replication of MPV is surprising, because Ribavirin does not work against all members of the Paramyxoviridae family.

For example, Nichols et al. (Blood 2001 volume 98, 3:573-578) and Elizaga et al. (Clin Infect Dis 2001, 32:413-418) reported that Ribavirin is not an effective agent against parainfluenza virus, which belongs to the same family as MPV and RSV. The study by Elizaga et al. involved treating 18 of 24 patients infected with PIV type 3 (PIV3) with a Ribavirin treatment starting only three days after the onset of symptoms. However, despite this early treatment with Ribavirin, no improvement in outcome was observed. In the study by Nichols et al. 31 of 55 patients with PIV3 pneumonia were treated with Ribavirin therapy within 48 hours of diagnosis. Also here, characteristics (viral shedding) of treated and untreated patients were similar and the 30-day mortality rate did not appear to be affected by the administration of Ribavirin. These data clearly indicate that Ribavirin is not active against all members of the paramyxovirus family, thereby underscoring the unexpected results shown in the present invention.

Ribavirin, although currently licensed for therapy of respiratory syncytial virus (RSV) pneumonia and bronchiolitis (Hall et al, N. Engl. J. Med., 308:1443 (1983); Hall et al., JAMA, 254: 3047 (1985), is still of controversial therapeutic value as it has to be administered over an 18 hour period by aerosol inhalation. In addition, the level of secondary infection following cessation of treatment is significantly higher than in untreated patients.

One effective approach to solve this problem as disclosed herein is to prepare a medicament comprising a therapeutically effective amount of a nucleoside analog with an antibody antimicrobial agent (e.g. anti-hMPV antibody). As disclosed herein the presence of the antibody antimicrobial agent not only substantially reduced the dosage of the nucleoside analog required to achieve the desired effect, but also enhanced the effectiveness of the nucleoside analog, without unwanted cross-reactions and other interfering effects.

Furthermore, viral infections with human metapneumovirus may be further complicated by secondary pathogenic infections, like SARS-associated coronavirus (SARS-CoV) infections and bacterial pneumonia. Thus for an effective treatment it would be desirable to threat both bacterial and viral infections simultaneously, using a combined approach. A combined approach as used herein, not only limits the undesirable effects of use of the above mentioned nucleoside analog, but is also extremely effective in preventing or treating respiratory infections, caused by mammalian MPV and associated secondary infections, which together up until the present time were untreatable.

### SUMMARY OF THE INVENTION

The invention provides the use of a nucleoside analog and an antimicrobial neutralising antibody for the manufacture of a medicament for treating or preventing respiratory tract infections in a subject infected with a mammalian MPV. In a preferred embodiment said nucleoside analog comprises Ribavirin or a derivative thereof. The invention further provides an antimicrobial neutralising antibody, wherein said antimicrobial neutralising antibody comprises an antiviral antibody, or derivative thereof. In a preferred embodiment said antiviral antibody comprises an anti-hMPV antibody.

In addition the invention provides the use of a nucleoside analog or a derivative thereof and an antimicrobial neutralising antibody or derivative thereof, for the manufacture of a medicament for treating or preventing respiratory tract infections in a subject infected with a mammalian MPV and co-infected with one or more viruses from the Paramyxoviridae family, such as RSV.

The invention further provides use of a nucleoside analog or a derivative thereof and antimicrobial neutralising antibody or derivative thereof, for the manufacture of a medicament for treating or preventing respiratory tract infections in a subject infected with mammalian MPV and co-infected with one or more other respiratory pathogen. For example, said nucleoside analog comprises Ribavirin or a derivative thereof and said antimicrobial neutralising antibody comprises an anti-hMPV antibody. In another preferred embodiment said respiratory tract infections, comprise viral lower respiratory tract infections. For instance, said subject is a human subject less than 5 years old, preferably less than 2 years old. Said human subject may be infected with the SARS-associated coronavirus (SARS-CoV) and suffering from severe acute respiratory syndrome (SARS). In another preferred embodiment of the present invention said medicament further comprises interferon, preferably interferon alpha-2B. Said subject may also be an animal, especially a mammal.

Furthermore the invention provides a method for treating or preventing respiratory tract infections in a subject infected with a mammalian MPV, said method comprising administering a nucleoside analog and an antimicrobial neutralising antibody to said subject. In addition the invention provides a method for treating or preventing respiratory tract infections in a subject infected with a mammalian MPV and co-infected with one or more viruses from the Paramyxoviridae family, said method comprising administering a nucleoside analog and an antimicrobial neutralising antibody to said subject. Preferably said subject is co-infected with a virus that belongs to the Pneumovirinae sub-family, even more preferred Respiratory Syncitial Virus (RSV).

Moreover the invention provides a method for treating or preventing respiratory tract infections in a subject infected with mammalian MPV and co-infected with one or more other respiratory pathogens, said method comprising administering a nucleoside analog and antimicrobial neutralising antibody to said subject. Also part of the invention is that said subject is co-infected with one or more other RNA viruses, preferably with a member of the Coronavirus family, more preferred with a SARS-related Coronavirus.

A preferred embodiment is a method according to the invention wherein said nucleoside analog comprises Ribavirin or a derivative thereof. In another preferred embodiment is a method according to the invention wherein said antimicrobial neutralising antibody comprises an anti-hMPV antibody. For instance, is provided a method according to the invention wherein said mammalian MPV is hMPV. It is understood that said respiratory tract infections may comprise viral lower respiratory tract infections. Preferably said subject is human, for example less than 5 years old, even more preferred said subject is less than 2 years old or said subject is elderly.

In another aspect the invention provides a method according to the invention, wherein the subject additionally suffers from a disease or condition other than a respiratory tract infection, preferably a disease or condition selected from a group essentially consisting of cystic fibrosis, non-Hodgkin lymphoma, asthma, bone marrow transplantation and kidney transplantation. As well is provided a method according to the invention, wherein the subject is immunocompromised. In particular said subject suffers from SARS.

### DESCRIPTION OF THE FIGURES

FIG. 1. Number of hMPV infected cells as determined by immune fluorescence. Five fields were counted under the microscope at a high power magnification (320×).

FIG. 2.

Percentage of cells infected with hMPV. Cells were infected with 30 TCID50 of hMPV and after 0, 2, 4, 8 and 16 hours, post-infection, 0, 5, 10, 25, 50 or 100 microgram of Ribavirin was added. Three days post-infection, cells were fixed and stained with a hMPV-specific polyclonal antibody, and the number of hMPV positive and negative cells were counted in 5 fields.

FIG. 3. Inhibition of hMPV replication in vitro by ribavirin and hMPV anti-serum. Cells were infected with either 50 or 250 TCID50 of virus and treated with different concentrations of ribavirin (see x-axis). Different bars indicate treatment with different concentrations of anti-serum.

### DETAILED DESCRIPTION OF THE INVENTION

In a preferred embodiment the invention provides the use of a nucleoside analog and an antimicrobial neutralising antibody for the manufacture of a medicament for treading or preventing respiratory tract infections in a subject infected with a mammalian MPV. In a preferred embodiment of the invention, said nucleoside analog is a guanosine analog. More preferred, said guanosine analog is 1-(5-Deoxy-[beta]-D-ribo-furanosyl)-1,2,4-triazole-3-carboxamide, also known as Ribavirin or Virazole, or a derivative thereof Herewith, we have identified the nucleoside analog Ribavirin as a prophylactic agent against a mammalian MPV. What is more, the analog is also capable of reducing the replication rate of MPV when administered to cells post-infection. As is exemplified herein, following infection with hMPV, cells were treated after different time intervals (0-16 hours) with various concentrations (0-100 microgram/ml) of Ribavirin. Staining of the cells for the presence of hMPV revealed that Ribavirin at a concentration of 25 microgram/ml or higher greatly reduced the percentage of MPV-positive cells 3 days post-infection.

Thus, in addition to the prophylactic effect against MPV, the nucleoside analog also has a therapeutic effect because it can decrease viral replication post-infection.

All antiviral nucleosides with mutagenicity similar to that of ribavirin which are capable of reducing the replication rate of MPV, having both a therapeutic and prophylactic effect against MPV, are deemed encompassed by the present invention. A nucleoside analog of the present invention can be used in combination with an antimicrobial neutralising antibody for the manufacture of a medicament to treat a patient infected with human MPV (hMPV). Anti-“microbial” as used herein refers to anti-“viral”, anti-“bacterial”, anti-“fungal” etc.

As used herein, the terms “antibody” and “antibodies” refer to monoclonal antibodies, multispecific antibodies (e.g., bi-specific), human antibodies, humanized antibodies, camelised antibodies, chimeric antibodies, single-chain Fvs (scFv), single chain antibodies, synthetic antibodies, single domain antibodies, Fab fragments, F(ab) fragments, disulfide-linked Fvs (sdFv), and anti-idiotypic (anti-Id) antibodies, and epitope-binding fragments of any of the above. In particular, antibodies include immunoglobulin molecules and immunologically active fragments of immunoglobulin molecules, ie., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁, and IgA₂) or subclass.

Antibodies of the invention include, but are not limited to, monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to an hMPV antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies of the invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

In certain embodiments, high potency antibodies can be used in the methods of the invention. For example, high potency antibodies can be produced by genetically engineering appropriate antibody gene sequences and expressing the antibody sequences in a suitable host. The antibodies produced can be screened to identify antibodies with, e.g., high kₒₙ values in a BIAcore assay (see section 4.8.3).

In certain embodiments, an antibody to be used with the methods of the present invention or fragment thereof has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody to be used with the methods of the invention or fragment thereof has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In certain embodiments, an antibody to be used with the methods of the invention or fragment thereof that has a median effective concentration (EC₅₀) of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay. The median effective concentration is the concentration of antibody or antibody fragments that neutralizes 50% of the hMPV in an in vitro microneutralization assay. In a preferred embodiment, an antibody to be used with the methods of the invention or fragment thereof has an EC₅₀ of less than 0.01 nM, less than 0.025 nM, less than 0.05 nM, less than 0.1 nM, less than 0.25 nM, less than 0.5 nM, less than 0.75 nM, less than 1 nM, less than 1.25 nM, less than 1.5 nM, less than 1.75 nM, or less than 2 nM, in an in vitro microneutralization assay.

The antibodies to be used with the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, synthesis in the presence of tunicamycin, etc. Additionally, the derivative may contain one or more non-classical amino acids.

The present invention also provides antibodies of the invention or fragments thereof that comprise a framework region known to those of skill in the art. In certain embodiments, one or more framework regions, preferably, all of the framework regions, of an antibody to be used in the methods of the invention or fragment thereof are human. In certain other embodiments of the invention, the fragment region of an antibody of the invention or fragment thereof is humanized. In certain embodiments, the antibody to be used with the methods of the invention is a synthetic antibody, a monoclonal antibody, an intrabody, a chimeric antibody, a human antibody, a humanized chimeric antibody, a humanized antibody, a glycosylated antibody, a multispecific antibody, a human antibody, a single-chain antibody, or a bispecific antibody.

In certain embodiments of the invention, the antibodies to be used with the invention have half-lives in a mammal, preferably a human, of greater than 12 hours, greater than 1 day, greater than 3 days, greater than 6 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. Antibodies or antigen-binding fragments thereof having increased in vivo half-lives can be generated by techniques known to those of skill in the art. For example, antibodies or antigen-binding fragments thereof with increased in vivo half-lives can be generated by modifying (e.g., substituting, deleting or adding) amino acid residues identified as involved in the interaction between the Fc domain and the FcRn receptor (see, e.g., PCT Publication No. WO 97/34631 and U.S. patent application Ser. No. 10/020,354, entitled “Molecules with Extended Half-Lives, Compositions and Uses Thereof”, filed Dec. 12, 2001, by Johnson et al., which are incorporated herein by reference in their entireties). Such antibodies or antigen-binding fragments thereof can be tested for binding activity to hMPV antigens as well as for in vivo efficacy using methods known to those skilled in the art, for example, by immunoassays described herein.

Further, antibodies or antigen-binding fragments thereof with increased in vivo half-lives can be generated be attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography. PEG-derivatizated antibodies or antigen-binding fragments thereof can be tested for binding activity to hMPV antigens as well as for in vivo efficacy using methods known to those skilled in the art, for example, by immunoassays described herein.

In certain embodiments, the antibodies to be used with the methods of the invention are fusion proteins comprising an antibody or fragment thereof that immunospecifically binds to an hMPV antigen and a heterologous polypeptide. Preferably, the heterologous polypeptide that the antibody or antibody fragment is flised to is useful for targeting the antibody to respiratory epithelial cells.

In certain embodiments, antibodies to be used with the methods of the invention or fragments thereof disrupt or prevent the interaction between an hMPV antigen and its host cell receptor.

In certain embodiments, antibodies to be used with the methods of the invention are single-chain antibodies. The design and construction of a single-chain antibody is described in Marasco et al, 1993, Proc Natl Acad Sci 90:7889-7893, which is incorporated herein by reference in its entirety.

In certain embodiments, the antibodies to be used with the invention binds to an intracellular epitope, i. e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). sFv are antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, Wash., 1998, “intrabodies: Basic Research and Clinical Gene Therapy Applications” Springer:New York).

Generation of intrabodies is well-known to the skilled artisan and is described for example in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies (e.g., Section 4.1.2 and 4.1.3) may also be used in the generation of intrabodies. A discussion of intrabodies as antiviral agents can also be found in Marasco, 2001, Curr. Top. Microbiol. Immunol. 260:247-270, which is incorporated by reference herein in its entirety.

In one embodiment, intrabodies of the invention retain at least about 75% of the binding effectiveness of the complete antibody (i.e., having constant as well as variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see example, Huston, et al., 1991, Methods in Enzym. 203:46-121, which is incorporated herein by reference). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assess the degree of antigen binding for each. Examples of linkers include, but are not limited to, those sequences disclosed in Table 1.

TABLE 1
Sequence

(Gly Gly Gly Gly Ser)₃
Glu Ser Gly Arg Ser Gly Gly Gly Gly Ser Gly Gly
Gly Gly Ser
Gln Gly Lys Ser Ser Gly Ser Gly Ser Glu Ser Lys
Ser Thr
Gln Gly Lys Ser Ser Gly Ser Gly Ser Glu Ser Lys
Ser Thr Gln
Glu Gly Lys Ser Ser Gly Ser Gly Ser Gln Ser Lys
Val Asp
Gly Ser Thr Ser Gly Ser Gly Lys Ser Ser Glu Gly
Lys Gly
Lys Glu Ser Gly Ser Val Ser Ser Gln Gln Leu Ala
Gln Phe Arg Ser Leu Asp
Glu Ser Gly Ser Val Ser Ser Gln Gln Leu Ala Phe
Arg Ser Leu Asp

In one embodiment, intrabodies are expressed in the cytoplasm. In other embodiments, the intrabodies are localized to various intracellular locations. In such embodiments, specific localization sequences can be atached to the intranucleotide polypepetide to direct the intrabody to a specific location. Intrabodies can be localized, for example, to the folowing intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J. Can. Res. 76:1132-5; Ratner et al., 1985, Nature 313:277-84). Examples of localization signals include, but are not limited to, those sequences disclosed in Table 2.

TABLE 2

| | |
|---|---|
| Localization | Sequence |

| | |
|---|---|
| endoplasmic reticulum | Lys Asp Glu Leu |
| endoplasmic reticulum | Asp Asp Glu Leu |
| endoplasmic reticulum | Asp Glu Glu Leu |
| endoplasmic reticulum | Gln Glu Asp Leu |
| endoplasmic reticulum | Arg Asp Glu Leu |
| nucleus | Pro Lys Lys Lys Arg Lys Val |
| nucleus | Pro Gln Lys Lys Ile Lys Ser |
| nucleus | Gln Pro Lys Lys Pro |
| nucleus | Arg Lys Lys Arg |
| nucleolar region | Arg Lys Lys Arg Arg Gln Arg |
| | Arg Arg Ala His Gln |
| nucleolar region | Arg Gln Ala Arg Arg Asn Arg |
| | Arg Arg Arg Trp Arg Glu Arg |
| | Gln Arg |
| nucleolar region | Met Pro Leu Thr Arg Arg Arg |
| | Pro Ala Ala Ser Gln Ala Leu |
| | Ala Pro Pro Thr Pro |
| endosomal | Met Asp Asp Gln Arg Asp Leu |
| | Ile |
| compartment | Ser Asn Asn Glu Gln Leu Pro |
| mitochondrial matrix | Met Leu Phe Asn Leu Arg Xaa |
| | Xaa Leu Asn Asn Ala Ala Phe |
| | Arg His Gly His Asn Phe Met |
| | Val Arg Asn Phe Arg Cys Gly |
| | Gln Pro Leu Xaa |
| plasma membrane | GCVCSSNP |
| plasma membrane | GQTVTTPL |
| plasma membrane | GQELSQHE |
| plasma membrane | GNSPSYNP |
| plasma membrane | GVSGSKGQ |
| plasma membrane | GQTITTPL |
| plasma membrane | GQTLTTPL |
| plasma membrane | GQIFSRSA |
| plasma membrane | GQIHGLSP |
| plasma membrane | GARASVLS |
| plasma membrane | GCTLSAEE |

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol Biol. 291:1129-34).

Methods for Producing Antibodies

The antibodies to be used with the methods of the invention or fragments thereof can be produced by any method known in the art for the synthesis of antibodies, in particular, by chemical synthesis or preferably, by recombinant expression techniques.

Polyclonal antibodies to an hMPV antigen can be produced by various procedures well known in the art. For example, an hMPV antigen can be administered to various host animals including, but not limited to, rabbits, mice, rats, etc. to induce the production of sera containing polyclonal antibodies specific for the hMPV antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an hMPV antigen and once an immune response is detected, e.g., antibodies specific for the hMPV antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

In a specific embodiment, an antigen of APV is used to generate antibodies agains hMPV.

In certain embodiments, a method of generating monoclonal antibodies comprises culturing a hybridoma cell secreting an antibody of the invention wherein, preferably, the hybridoma is generated by fusing splenocytes isolated from a mouse immunized with an hMPV antigen with myeloma cells and then screening the hybridomas resulting from the fusion for hybridoma clones that secrete an antibody able to bind an hMPV antigen.

Antibody fragments which recognize specific hMPV epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies to be used with the present invention can also be generated using various phage display methods known in the art.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an hMPV antigen of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies an be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous iunmunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entireties. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human (e.g., murine) antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entireties. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332). In a preferred embodiment, antibodies comprise one or more CDRs listed in Table 3 (preferably all CDRs) and human framework regions. Often, fiamework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies to be used with the methods of the invention can, in turn, be utilized to generate anti-idiotype antibodies that “mimic” hMPV antigens using techniques well known to those skilled in the art. (See, e.g., Greenspan & Bona, 1989, FASEB J. 7(5):437-444; and Nissinoff, 1991, J. Immunol. 147(8):2429-2438). For example, antibodies of the invention which bind to and competitively inhibit the binding of hMPV (as determined by assays well known in the art) to its host cell receptor can be used to generate anti-idiotypes that “mimic” an hMPV antigen and bind to the hMPV receptors, i.e., compete with the virus for binding to the host cell, therefore decreasing the infection rate of host cells with virus.

In certain other embodiments, anti-anti-idiotypes, generated by techniques well-known to the skilled artisan, are used in the methods of the invention. Such anti-anti-idiotypes mimic the binding domain of the anti-hMPV antibody and, as a consequence, bind to and neutralize hMPV. Such neutralizing anti-anti-idiotypes or Fab fragments of such anti-anti-idiotypes can be used in therapeutic regimens to neutralize hMPV. For example, such anti-anti-idiotypic antibodies can be used to bind hMPV and thereby prevent infection.

In certain embodiments, a fragment of a protein of hMPV is used as an immunogen for the generation of antibodies to be used with the methods of the invention. A fragment of a protein of hMPV to be used as an immunogen can be at least 10, 20, 30, 40, 50, 75, 100, 250, 500, 750, or at least 1000 amino acids in length. In certain embodiments a synthetic peptide of a protein of hMPV is used as an immunogen.

Polynucleotides Encoding an Antibody

Polynucleotides encoding antibodies to be used with the invention may be obtained, and the nucleotide sequence of the polynucleotides determined, by any method known in the art. Since amino acid sequences of some antibodies are known (as described in Table 2), nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to an hMPV antigen. In certain embodiments, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Recobinant Expression of an Antibody

Recombinant expression of an antibody to be used with the methods of the invention, derivative or analog thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the apropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies or antigen-binding fragments thereof which immunospecifically bind to one or more hMPV antigens is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1995, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa califomica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197). The coding sequences for the heavy and light chains may comprise cDNA or genomic DNA.

Once an antibody molecule to be used with the methods of the invention has been produce by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

BiTE Technology

In certain embodiments, antibodies to be used with the methods of the invention and antibodies of the pharmaceutical compositions of the invention are bispecific T cell engagers (BiTEs). Bispecific T cell engagers (BiTE) are bispecific antibodies that can redirect T cells for antigen-specific elimination of targets. A BiTE molecule has an antigen-binding domain that binds to a T cell antigen (e.g. CD3) at one end of the molecule and an antigen binding domain that will bind to an antigen on the target cell. A BiTE molecule was recently described in WO 99/54440, which is herein incorporated by reference. This publication describes a novel single-chain multifunctional polypeptide that comprises binding sites for the CD19 and CD3 antigens (CD19×CD3). This molecule was derived from two antibodies, one that binds to CD19 on the B cell and an antibody that binds to CD3 on the T cells. The variable regions of these different antibodies are linked by a polypeptide sequence, thus creating a single molecule. Also described, is the linking of the variable heavy chain (VH) and light chain (VL) of a specific binding domain with a flexible linker to create a single chain, bispecific antibody.

In an embodiment of this invention, an antibody or a fragment thereof that immunospecifically binds a polypeptide of interest (e.g., an antigen of MPV) will comprise a portion of the BiTE molecule. For example, the VH and/or VL (preferably a scFV) of an antibody that binds a polypeptide of interest (e.g., an antigen of MPV) can be fused to an anti-CD3 binding portion such as that of the molecule described above, thus creating a BiTE molecule that targets the polypeptide of interest (e.g., an antigen of MPV). In addition to the variable heavy and or light chain of antibody against a polypeptide of interest (e.g., an antigen of MPV), other molecules that bind the polypeptide of interest (e.g., an antigen of MPV) can comprise the BiTE molecule, for example antiviral compounds. In another embodiment, the BiTE molecule can comprise a molecule that binds to other T cell antigens (other than CD3). For example, ligands and/or antibodies that immunospecifically bind to T-cell antigens like CD2, CD4, CD8, CD11a, TCR, and CD28 are contemplated to be part of this invention. This list is not meant to be exhaustive but only to illustrate that other molecules that can immunospecifically bind to a T cell antigen can be used as part of a BiTE molecule. These molecules can include the VH and/or VL portions of the antibody or natural ligands (for example LFA3 whose natural ligand is CD3). A BiTE molecule can be an antagonist.

The “binding domain” as used in accordance with the present invention denotes a domain comprising a three-dimensional structure capable of specifically binding to an epitope like native antibodies, free scFv fragments or one of their corresponding immunoglobulin chains, preferably the VH chain. Thus, said domain can comprise the VH and/or VL domain of an antibody or an immunoglobulin chain, preferably at least the VH domain or more preferably the VH and VL domain linked by a flexible polypeptide linker (scFv). On the other hand, said binding domain contained in the polypeptide of interest may comprise at least one complementarity determining region (CDR) of an antibody or immunoglobulin chain recognizing an antigen on the T cell or a cellular antigen. In this respect, it is noted that the binding domain present in the polypeptide of interest may not only be derived from antibodies but also from other T cell or cellular antigen binding protein, such as naturally occurring surface receptors or ligands. It is further contemplated that in an embodiment of the invention, said first and or second domain of the above-described polypeptide mimic or correspond to a VH and VL region from a natural antibody. The antibody providing the binding site for the polypeptide of interest can be, e.g., a monoclonal antibody, polyclonal antibody, chimeric antibody, humanized antibody, bispecific antibody, synthetic antibody, antibody fragment, such as Fab, Fv or scFv fragments etc., or a chemically modified derivative of any of these.

Antibody Conjugates

In certain embodiments, the antibodies to be used with the methods of the invention or fragments thereof are recombinantly fused or chemically conjugated (including both covalently and non-covalently conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. For example, antibodies may be used to target heterologous polypeptides to particular cell types (e.g., respiratory epithelial cells), either in vitro or in vivo, by fusing or conjugating the antibodies to antibodies specific for particular cell surface receptors. Antibodies fused or conjugated to heterologous polypeptides may also be used in in vitro immunoassays and purification methods using methods known in the art. See e.g., PCT publication WO 93/21232; EP 439,095; Naramura et al., Immunol. Lett. 39:91-99 (1994); U.S. Pat. No. 5,474,981; Gillies et al., PNAS 89:1428-1432 (1992); and Fell et al., J. Immunol. 146:2446-2452 (1991), which are incorporated by reference in their entireties.

In certain embodiments, the anti-hMPV-antigen antibody is an antibody conjugate.

Additional fusion proteins of the antibodies to be used with the methods of the invention or fragments thereof may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or antigen-binding fragments thereof with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, antibodies or antigen-binding fragments thereof, or the encoded antibodies or antigen-binding fragments thereof, may be altered by being subjected to random mutagenesis by error-prone PCR, random nucleotide insertion or other methods prior to recombination. In another embodiment, one or more portions of a polynucleotide encoding an antibody or antibody fragment, which portions immunospecifically bind to an hMPV antigen may be recombined with one or more components, motifs, sections, parts, domains, fragments, etc. of one or more heterologous molecules.

Moreover, the antibodies to be used with the methods of the present invention or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989 Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

An antibody or fragment thereof may be conjugated to a therapeutic moiety such as, but not limited to, a cytotoxin, e.g., a cytostatic or cytocidal agent, a therapeutic agent or a radioactive metal ion, e.g., alpha-emitters. A cytotoxin or cytotoxic agent includes, but is not limited to, any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), and antivirals, such as, but not limited to: nucleoside analogs, such as zidovudine, acyclovir, gangcyclovir, vidarabine, idoxuridine, trifluridine, and ribavirin, as well as foscamet, amantadine, rimantadine, saquinavir, indinavir, ritonavir, and the alpha-interferons.

Further, an antibody to be used with the methods of the invention or fragment thereof may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, but are not limited to, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Anti-HMPV-Antigen Antibodies

Any antibody that immunospecifically binds to an hMPV or to a protein of hMPV or a fragment, an analog, a derivative or a homolog thereof can be used with the methods of the invention.

hMPV

Structural Characteristics of a Mammalian Metapneumovirus

A Mammalian MPV is a negative-sense single stranded RNA virus belonging to the sub-family Pneumovirinae of the family Paramyxoviridae. Moreover, the mammalian MPV is identifiable as phylogenetically corresponding to the genus Metapneumovirus, wherein the mammalian MPV is phylogenetically more closely related to a virus isolate deposited as I-2614 with CNCM, Paris (SEQ ID NO:19) than to turkey rhinotracheitis virus, the etiological agent of avian rhinotracheitis. A virus is identifiable as phylogenetically corresponding to the genus Metapneumovirus by, e.g., obtaining nucleic acid sequence information of the virus and testing it in phylogenetic analyses. Any technique known to the skilled artisan can be used to determine phylogenetic relationships between strains of viruses. Other techniques are disclosed in International Patent Application PCT/NL02/00040, published as WO 02/057302, which is incorporated by reference in its entirety herein. In particular, PCT/NL02/00040 discloses nucleic acid sequences that are suitable for phylogenetic analysis at page 12, line 27 to page 19, line 29, which are incorporated by reference herein. A virus can further be identified as a mammalian MPV on the basis of sequence similarity as described in more detail below.

In a specific embodiment, the mammalian MPV is a human MPV.

In addition to phylogenetic relatedness and sequence similarity of a virus to a mammalian MPV as disclosed herein, the similarity of the genomic organization of a virus to the genomic organization of a mammalian MPV disclosed herein can also be used to identify the virus as a mammalian MPV. In certain embodiments, the genomic organization of a mammalian MPV is different from the genomic organization of pneumoviruses within the sub-family Pneumovirinae of the family Paramyxoviridae. The classification of the two genera, metapneumovirus and pneumovirus, is based primarily on their gene constellation; metapneumoviruses generally lack non-structural proteins such as NS1 or NS2 (see also Randhawa et al., 1997, J. Virol. 71:9849-9854) and the gene order is different from that of pneumoviruses (RSV: ‘3-NS1-NS2-N-P-M-SH-G-F-M2-L-5’, APV: ‘3-N-P-M-F-M2-SH-G-L-5’) (Lung, et al., 1992, J. Gen. Virol. 73:1709-1715; Yu, et al., 1992, Virology 186:426-434; Randhawa, et al., 1997, J. Virol. 71:9849-9854).

Further, a mammalian MPV of the invention can be identified by its immunological properties. In certain embodiments, specific anti-sera can be raised against mammalian MPV that can neutralize mammalian MPV. Monoclonal and polyclonal antibodies can be raised against MPV that can also neutralize mammalian MPV. (See, WO 02/057302, which is incorporated by reference herein.

The mammalian MPV of the invention is further characterized by its ability to infect a mammalian host, i.e., a mammalian cultured cell or a mammal. Unlike APV, mammalian MPV does not replicate or replicates only at low levels in chickens and turkeys. Mammalian MPV replicates, however, in mammalian hosts, such as cynomolgous macaques. In certain, more specific, embodiments, a mammalian MPV is further characterized by its ability to replicate in a mammalian host. In certain, more specific embodiments, a mammalian MPV is further characterized by its ability to cause the mammalian host to express proteins encoded by the genome of the mammalian MPV. In even more specific embodiments, the viral proteins expressed by the mammalian MPV are inserted into the cytoplasmic membranes of the mammalian host. In certain embodiments, the mammalian MPV of the invention can infect a mammalian host and cause the mammalian host to produce new infectious viral particles of the mammalian MPV. For a more detailed description of the functional characteristics of the mammalian MPV of the invention, see below.

In certain embodiments, the appearance of a virus in an electron microscope or its sensitivity to chloroform can be used to identify the virus as a mammalian MPV. The mammalian MPV of the invention appears in an electron microscope as paramyxovirus-like particle. Consistently, a mammalian MPV is sensitive to treatment with chloroform; a mammalian MPV is cultured optimally on tMK cells or cells functionally equivalent thereto and it is essentially trypsine dependent in most cell cultures. Furthermore, a mammalian MPV has a typical cytopathic effects (CPE) and lacks haemagglutinating activity against species of red blood cells. The CPE induced by MPV isolates are similar to the CPE induced by hRSV, with characteristic syncytia formation followed by rapid internal disruption of the cells and subsequent detachment from the culture plates. Although most paramyxoviruses have haemagglutinating activity, most of the pneumoviruses do not (Pringle, C. R. In: The Paramyxoviruses; (ed. D. W. Kingsbury) 1-39 (Plenum Press, New York, 1991)). A mammalian MPV contains a second overlapping ORF (M2-2) in the nucleic acid fragment encoding the M2 protein. The occurrence of this second overlapping ORF occurs in other pneumoviruses as shown in Ahmadian et al., 1999, J. Gen. Vir. 80:2011-2016.

In certain embodiments, a viral isolate can be identified as a mammalian MPV by the following method. A test sample can, e.g., be obtained from an animal or human. The sample is then tested for the presence of a virus of the sub-family Pneumovirinae. If a virus of the sub-family Pneumovirinae is present, the virus can be tested for any of the characteristics of a mammalian MPV as discussed herein, such as, but not limited to, phylogenetic relatedness to a mammalian MPV, nucleotide sequence identity to a nucleotide sequence of a mammalian MPV, amino acid sequence identity/homology to a amino acid sequence of a mammalian MPV, and genomic organization. Furthermore, the virus can be identified as a mammalian MPV by cross-hybridization experiments using nucleic acid sequences from a MPV isolate, RT-PCR using primers specific to mammalian MPV, or in classical cross-serology experiments using antibodies directed against a mammalian MPV isolate. In certain other embodiments, a mammalian MPV can be identified on the basis of its immunological distinctiveness, as determined by quantitative neutralization with animal antisera. The antisera can be obtained from, e.g., ferrets, pigs or macaques that are infected with a mammalian MPV.

In certain embodiments, the serotype does not cross-react with viruses other than mammalian MPV. In other embodiments, the serotype shows a homologous-to-heterologous titer ratio >16 in both directions. If neutralization shows a certain degree of cross-reaction between two viruses in either or both directions (homologous-to-heterologous titer ration of eight or sixteen), distinctiveness of serotype is assumed if substantial biophysical/biochemical differences of DNA sequences exist. If neutralization shows a distinct degree of cross-reaction between two viruses in either or both directions (homologous-to-heterologous titer ratio of smaller than eight), identity of serotype of the isolates under study is assumed. Isolate I-2614, herein also known as MPV isolate 00-1 (as deposited with CNCM, Paris (SEQ ID NO:19)), can be used as prototype.

In certain embodiments, a virus can be identified as a mammalian MPV by means of sequence homology/identity of the viral proteins or nucleic acids in comparison with the amino acid sequence and nucleotide sequences of the viral isolates disclosed herein by sequence or deposit. In particular, a virus is identified as a mammalian MPV when the genome of the virus contains a nucleic acid sequence that has a percentage nucleic acid identity to a virus isolate deposited as I-2614 with CNCM, Paris which is higher than the percentages identified herein for the nucleic acids encoding the L protein, the M protein, the N protein, the P protein, or the F protein as identified herein below in comparison with APV-C (see Table 4). (See, PCT WO 02/05302, at pp. 12 to 19, which is incorporated by reference herein. Without being bound by theory, it is generally known that viral species, especially RNA virus species, often constitute a quasi species wherein the members of a cluster of the viruses display sequence heterogeneity. Thus, it is expected that each individual isolate may have a somewhat different percentage of sequence identity when compared to APV-C.

The highest amino sequence identity between the proteins of MPV and any of the Icnown other viruses of the same family to date is the identity between APV-C and human MPV. Between human MPV and APV-C, the amino acid sequence identity for the matrix protein is 87%, 88% for the nucleoprotein, 68% for the phosphoprotein, 81% for the fusion protein and 56-64% for parts of the polymerase protein, as can be deduced when comparing the sequences given in FIG. 30, see also Table 4. Viral isolates that contain ORFs that encode proteins with higher homology compared to these maximum values are considered mammalian MPVs. It should be noted that, similar to other viruses, a certain degree of variation is found between different isolated of mammalian MPVs.

TABLE 4
Amino acid sequence identity between the ORFs
of MPV and those of other paramyxoviruses.

| | N | P | M | F | M2-1 | M2-2 | L |
|---|---|---|---|---|---|---|---|
| APV A | 69 | 55 | 78 | 67 | 72 | 26 | 64 |
| APV B | 69 | 51 | 76 | 67 | 71 | 27 | —² |
| APV C | 88 | 68 | 87 | 81 | 84 | 56 | —² |
| hRSVA | 42 | 24 | 38 | 34 | 36 | 18 | 42 |
| hRSV B | 41 | 23 | 37 | 33 | 35 | 19 | 44 |
| bRSV | 42 | 22 | 38 | 34 | 35 | 13 | 44 |
| PVM | 45 | 26 | 37 | 39 | 33 | 12 | —² |
| others³ | 7-11 | 4-9 | 7-10 | 10-18 | —⁴ | —⁴ | 13-14 |

Footnotes:
¹No sequence homologies were found with known G and SH proteins and were thus excluded
²Sequences not available.
³others: human parainfluenza virus type 2 and 3, Sendai virus, measles virus, nipah virus, phocine distemper virus, and New Castle Disease virus.
⁴ORF absent in viral genome.

Any protein of a mammalian MPV can be used as an immunogen to generate antibodies to be used with the methods of the invention. In certain embodiments, an antibody to be used with the methods of treatment of the present invention bind immunospecifically to a protein of mammlian MPV as set forth below.

In certain embodiments, the amino acid sequence of the SH protein of the mammalian MPV is at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:382 (SH protein of isolate NL/1/00; see Table 5). The isolated negative-sense single stranded RNA metapneumovirus that comprises the SH protein that is at least 30% identical to SEQ ID NO:382 (SH protein of isolate NL/1/00; see Table 5) is capable of infecting a mammalian host. In certain embodiments, the isolated negative-sense single stranded RNA metapneumovirus that comprises the SH protein that is at least 30% identical to SEQ ID NO:382 (SH protein of isolate NL/1/00; see Table 5) is capable of replicating in a mammalian host. In certain embodiments, a mammalian MPV contains a nucleotide sequence that encodes a SH protein that is at least 30% identical to SEQ ID NO:382 (SH protein of isolate NL/1/00; see Table 5).

In certain embodiments, the amino acid sequence of the G protein of the mammalian MPV is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or at least 99.5% identical to the amino acid sequence of SEQ ID NO:322 (G protein of isolate NUL/100; see Table 5). The isolated negative-sense single stranded RNA metapneumovirus that comprises the G protein that is at least 20% identical to SEQ ID NO:322 (G protein of isolate NL/1/00; see Table 5) is capable of infecting a mammalian host. In certain embodiments, the isolated negative-sense single stranded RNA metapneumovirus that comprises the G protein that is at least 20% identical to SEQ ID NO:322 (G protein of isolate NL/1/00; see Table 5) is capable of replicating in a mammalian host. In certain embodiments, a mammalian MPV contains a nucleotide sequence that encodes a G protein that is at least 20% identical to SEQ ID NO:322 (G protein of isolate NL/1/00; see Table 5).

In certain embodiments, the amino acid sequence of the L protein of the mammalian MPV is at least 85%, at least 90%, at least 95%, at least 98%, at least 99%, or at least 99.5% identical to the amino acid sequence of SEQ ID NO:330 (L protein of isolate NL/1/00; see Table 5). The isolated negative-sense single stranded RNA metapneumovirus that comprises the L protein that is at least 85% identical to SEQ ID NO:330 (L protein of isolate NL/1/00; see Table 5) is capable of infecting a mammalian host. In certain embodiments, the isolated negative-sense single stranded RNA metapneumovirus that comprises the L protein that is at least 85% identical to SEQ ID NO:330 (L protein of isolate NL/1/00; see Table 5) is capable of replicating in a mammalian host. In certain embodiments, a mammalian MPV contains a nucleotide sequence that encodes a L protein that is at least 20% identical to SEQ ID NO:330 (L protein of isolate NL/1/00; see Table 5).

In certain embodiments, the amino acid sequence of the N protein of the mammalian MPV is at least 90%, at least 95%, or at least 98% identical to the amino acid sequence of SEQ ID NO:366. The isolated negative-sense single stranded RNA metapneumovirus that comprises the N protein that is at least 90% identical in amino acid sequence to SEQ ID NO:366 is capable of infecting mammalian host. In certain embodiments, the isolated negative-sense single stranded RNA metapneumovirus that comprises the N protein that is 90% identical in amino acid sequence to SEQ ID NO:366 is capable of replicating in a mammalian host. The amino acid identity is calculated over the entire length of the N protein. In certain embodiments, a mammalian MPV contains a nucleotide sequence that encodes a N protein that is at least 90%, at least 95%, or at least 98% identical to the amino acid sequence of SEQ ID NO:366.

The amino acid sequence of the P protein of the mammalian MPV is at least 70%, at least 80%, at least 90%, at least 95% or at least 98% identical to the amino acid sequence of SEQ ID NO:374. The mammalian MPV that comprises the P protein that is at least 70% identical in amino acid sequence to SEQ ID NO:374 is capable of infecting a mammalian host. In certain embodiments, the mammalian MPV that comprises the P protein that is at least 70% identical in amino acid sequence to SEQ ID NO:374 is capable of replicating in a mammalian host. The amino acid identity is calculated over the entire length of the P protein. In certain embodiments, a mammalian MPV contains a nucleotide sequence that encodes a P protein that is at least 70%, at least 80%, at least 90%, at least 95% or at least 98% identical to the amino acid sequence of SEQ ID NO:374.

The amino acid sequence of the M protein of the mammalian MPV is at least 90%, at least 95% or at least 98% identical to the amino acid sequence of SEQ ID NO:358. The mammalian MPV that comprises the M protein that is at least 90% identical in amino acid sequence to SEQ ID NO:358 is capable of infecting mammalian host. In certain embodiments, the isolated negative-sense single stranded RNA metapneumovirus that comprises the M protein that is 90% identical in amino acid sequence to SEQ ID NO:358 is capable of replicating in a mammalian host. The amino acid identity is calculated over the entire length of the M protein. In certain embodiments, a mammalian MPV contains a nucleotide sequence that encodes a M protein that is at least 90%, at least 95% or at least 98% identical to the amino acid sequence of SEQ ID NO:358.

The amino acid sequence of the F protein of the mammalian MPV is at least 85%, at least 90%, at least 95% or at least 98% identical to the amino acid sequence of SEQ ID NO:314. The mammalian MPV that comprises the F protein that is at least 85% identical in amino acid sequence to SEQ ID NO:314 is capable of infecting a mammalian host. In certain embodiments, the isolated negative-sense single stranded RNA metapneumovirus that comprises the F protein that is 85% identical in amino acid sequence to SEQ ID NO:314 is capable of replicating in mammalian host. The amino acid identity is calculated over the entire length of the F protein. In certain embodiments, a mammalian MPV contains a nucleotide sequence that encodes a F protein that is at least 85%, at least 90%, at least 95% or at least 98% identical to the amino acid sequence of SEQ ID NO:314.

The amino acid sequence of the M2-1 protein of the mammalian MPV is at least 85%, at least 90%, at least 95% or at least 98% identical to the amino acid sequence of SEQ ID NO:338. The mammalian MPV that comprises the M2-1 protein that is at least 85% identical in amino acid sequence to SEQ ID NO:338 is capable of infecting a mammalian host. In certain embodiments, the isolated negative-sense single stranded RNA metapneumovirus that comprises the M2-1 protein that is 85% identical in amino acid sequence to SEQ ID NO:338 is capable of replicating in a mammalian host. The amino acid identity is calculated over the entire length of the M2-1 protein. In certain embodiments, a mammalian MPV contains a nucleotide sequence that encodes a M2-1 protein that is at least 85%, at least 90%, at least 95% or at least 98% identical to the amino acid sequence of SEQ ID NO:338.

The amino acid sequence of the M2-2 protein of the mammalian MPV is at least 60%, at least 70%, at least 80%, at least 90%, at least 95% or at least 98% identical to the amino acid sequence of SEQ ID NO:346 The isolated mammalian MPV that comprises the M2-2 protein that is at least 60% identical in amino acid sequence to SEQ ID NO:346 is capable of infecting mammalian host. In certain embodiments, the isolated negative-sense single stranded RNA metapneumovirus that comprises the M2-2 protein that is 60% identical in amino acid sequence to SEQ ID NO:346 is capable of replicating in a mammalian host. The amino acid identity is calculated over the entire length of the M2-2 protein. In certain embodiments, a mammalian MPV contains a nucleotide sequence that encodes a M2-1 protein that is is at least 60%, at least 70%, at least 80%, at least 90%, at least 95% or at least 98% identical to the amino acid sequence of SEQ ID NO:346.

In certain embodiments, the negative-sense single stranded RNA metapneumovirus encodes at least two proteins, at least three proteins, at least four proteins, at least five proteins, or six proteins selected from the group consisting of (i) a N protein with at least 90% amino acid sequence identity to SEQ ID NO:366; (ii) a P protein with at least 70% amino acid sequence identity to SEQ ID NO:374 (iii) a M protein with at least 90% amino acid sequence identity to SEQ ID NO:358 (iv) a F protein with at least 85% amino acid sequence identity to SEQ ID NO:314 (v) a M2-1 protein with at least 85% amino acid sequence identity to SEQ ID NO:338; and (vi) a M2-2 protein with at least 60% amino acid sequence identity to SEQ ID NO:346.

Mammalian MPV, can be divided into two subgroups, subgroup A and subgroup B, and the two subgroups can each be divided into two variants, A1 and A2, and B1 and B2. A mammalian MPV can be identified as a member of subgroup A if it is phylogenetically closer related to the isolate 00-1 (SEQ ID NO:19) than to the isolate 99-1 (SEQ ID NO:18). A mammalian MPV can be identified as a member of subgroup B if it is phylogenetically closer related to the isolate 99-1 (SEQ ID NO:18) than to the isolate 00-1 (SEQ ID NO:19). In other embodiments, nucleotide or amino acid sequence homologies of individual ORFs can be used to classify a mammalian MPV as belonging to subgroup A or B.

The different isolates of mammalian MPV can be divided into four different variants, variant A1, variant A2, variant B1 and variant B2 (see FIGS. 21 and 22). The isolate 00-1 (SEQ ID NO:19) is an example of the variant A1 of mammalian MPV. The isolate 99-1 (SEQ ID NO:18) is an example of the variant B1 of mammalian MPV. A mammalian MPV can be grouped into one of the four variants using a phylogenetic analysis. Thus, a mammalian MPV belongs to a specific variant if it is phylogenetically closer related to a known member of that variant than it is phylogenetically related to a member of another variant of mammalian MPV. The sequence of any ORF and the encoded polypeptide may be used to type a MPV isolate as belonging to a particular subgroup or variant, including N, P, L, M, SH, G, M2 or F polypeptides. In a specific embodiment, the classification of a mammalian MPV into a variant is based on the sequence of the G protein. Without being bound by theory, the G protein sequence is well suited for phylogenetic analysis because of the high degree of variation among G proteins of the different variants of mammalian MPV.

In certain embodiments of the invention, sequence homology may be determined by the ability of two sequences to hybridize under certain conditions, as set forth below. A nucleic acid which is hybridizable to a nucleic acid of a mammalian MPV, or to its reverse complement, or to its complement can be used in the methods of the invention to determine their sequence homology and identities to each other. In certain embodiments, the nucleic acids are hybridized under conditions of high stringency.

It is well-known to the skilled artisan that hybridization conditions, such as, but not limited to, temperature, salt concentration, pH, formamide concentration (see, e.g., Sambrook et al., 1989, Chapters 9 to 11, Molecular Cloning, A Laboratory Manual, 2d Ed., Cold Spring Harbor Laboratory Press, Cold Spring Harbor, N.Y., incorporated herein by reference in its entirety). In certain embodiments, hybridization is performed in aqueous solution and the ionic strength of the solution is kept constant while the hybridization temperature is varied dependent on the degree of sequence homology between the sequences that are to be hybridized. For DNA sequences that 100% identical to each other and are longer than 200 basepairs, hybridization is carried out at approximately 15-25° C. below the melting temperature (Tm) of the perfect hybrid. The melting temperature (Tm) can be calculated using the following equation (Bolton and McCarthy, 1962, Proc. Natl. Acad. Sci. USA 84:1390):
Tm=81.5° C.−16.6(log 10[Na+])+(%G+C)−0.63(%formamide)−(600/1)

Wherein (Tm) is the melting temperature, [Na+] is the sodium concentration, G+C is the Guanine and Cytosine content, and 1 is the length of the hybrid in basepairs. The effect of mismatches between the sequences can be calculated using the formula by Bonner et al. (Bonner et al., 1973, J. Mol. Biol. 81:123-135): for every 1% of mismatching of bases in the hybrid, the melting temperature is reduced by 1-1.5° C.

Thus, by determining the temperature at which two sequences hybridize, one of skill in the art can estimate how similar a sequence is to a known sequence. This can be done, e.g., by comparison of the empirically determined hybridization temperature with the hybridization temperature calculated for the know sequence to hybridize with its perfect match. Through the use of the formula by Bonner et al., the relationship between hybridization temperature and percent mismatch can be exploited to provide information about sequence similarity.

By way of example and not limitation, procedures using such conditions of high stringency are as follows. Prehybridization of filters containing DNA is carried out for 8 h to overnight at 65 C in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65 C in prehybridization mixture containing 100 μg/ml denatured salmon sperm DNA and 5-20×106 cpm of 32P-labeled probe. Washing of filters is done at 37 C for 1 h in a solution containing 2×SSC, 0.01% PVP, 0.01% Ficoll, and 0.01% BSA. This is followed by a wash in 0.1×SSC at 50 C for 45 min before autoradiography. Other conditions of high stringency which may be used are well known in the art. In other embodiments of the invention, hybridization is performed under moderate of low stringency conditions, such conditions are well-known to the skilled artisan (see e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc., each of which is incorporated by reference herein in their entirety). An illustrative low stringency condition is provided by the following system of buffers: hybridization in a buffer comprising 35% formamide, 5×SSC, 50 mM Tris-HCl (pH 7.5), 5 mM EDTA, 0.02% PVP, 0.02% Ficoll, 0.2% BSA, 100 μg/ml denatured salmon sperm DNA, and 10% (wt/vol) dextran sulfate for 18-20 hours at 4□C, washing in a buffer consisting of 2×SSC, 25 mM Tris-HCl (pH 7.4), 5 mM EDTA, and 0.1% SDS for 1.5 hours at 55□C, and washing in a buffer consisting of 2×SSC, 25 mM Tris-HCl (pH 7.4), 5 mM EDTA, and 0.1% SDS for 1.5 hours at 60□C.

In certain embodiments, a mammalian MPV can be classified into one of the variant using probes that are specific for a specific variant of mammalian MPV. Such probes include primers for RT-PCR (Table 5) and antibodies.

In certain embodiments of the invention, the different variants of mammalian MPV can be distinguished from each other by way of the amino acid sequences of the different viral proteins. In other embodiments, the different variants of mammalian MPV can be distinguished from each other by way of the nucleotide sequences of the different ORFs encoded by the viral genome. A variant of mammalian MPV can be, but is not limited to, A1, A2, B1 or B2.

An isolate of mammalian MPV is classified as a variant B1 if it is phylogenetically closer related to the viral isolate NL/1/99 (SEQ ID NO:18) than it is related to any of the following other viral isolates: NL/1/00 (SEQ ID NO:19), NL/17/00 (SEQ ID NO:20) and NL/1/94 (SEQ ID NO:21). One or more of the ORFs of a mammalian MPV can be used to classify the mammalian MPV into a variant. A mammalian MPV can be classified as an MPV variant B1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B1 as represented by the prototype NL/1/99 (SEQ ID NO:324); if the amino acid sequence of its N proteint is at least 98.5% or at least 99% or at least 99.5% identical to the N protein of a mammalian MPV variant B1 as represented by the prototype NL/1/99 (SEQ ID NO:368); if the amino acid sequence of its P protein is at least 96%, at least 98%, or at least 99% or at least 99.5% identical to the P protein of a mammalian MPV variant B1 as represented by the prototype NL/1/99 (SEQ ID NO:376); if the amino acid sequence of its M protein is identical to the M protein of a mammalian MPV variant B1 as represented by the prototype NL/1/99 (SEQ ID NO:360); if the amino acid sequence of its F protein is at least 99% identical to the F protein of a mammalian MPV variant B1 as represented by the prototype NL/1/99 (SEQ ID NO:316); if the amino acid sequence of its M2-1 protein is at least 98% or at least 99% or at least 99.5% identical to the M2-1 protein of a mammalian MPV variant B1 as represented by the prototype NL/1/99 (SEQ ID NO:340); if the amino acid sequence of its M2-2 protein is at least 99%or at least 99.5% identical to the M2-2 protein of a mammalian MPV variant B1 as represented by the prototype NL/1/99 (SEQ ID NO:348); if the amino acid sequence of its SH protein is at least 83%, at least 85%, at least 90%, at least 95%, at least 98%, or at least 99% or at least 99.5% identical to the SH protein of a mammalian MPV variant B1 as represented by the prototype NL/1/99 (SEQ ID NO:384); and/or if the amino acid sequence of its L protein is at least 99% or at least 99.5% identical to the L protein a mammalian MPV variant B1 as represented by the prototype NL/1/99 (SEQ ID NO:332).

An isolate of mammalian MPV is classified as a variant A1 if it is phylogenetically closer related to the viral isolate NL/1/00 (SEQ ID NO:19) than it is related to any of the following other viral isolates: NL/1/99 (SEQ ID NO:18), NL/17/00 (SEQ ID NO:20) and NL/1/94 (SEQ ID NO:21). One or more of the ORFs of a mammalian MPT can be used to classify the mammalian MPV into a variant. A mammalian MPV can be classified as an MPV variant A1, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant A1 as represented by the prototype NL/1/00 (SEQ ID NO:322); if the amino acid sequence of its N protein is at least 99.5% identical to the N protein of a mammalian MPV variant A1 as represented by the prototype NL/1/00 (SEQ ID NO:366); if the amino acid sequence of its P protein is at least 96%, at least 98%, or at least 99% or at least 99.5% identical to the P protein of a mammalian MPV variant A 1 as represented by the prototype NL/1/00 (SEQ ID NO:374); if the amino acid sequence of its M protein is at least 99% or at least 99.5% identical to the M protein of a mammalian MPV variant A1 as represented by the prototype NL/1/00 (SEQ ID NO:358); if the amino acid sequence of its F protein is at least 98% or at least 99% or at least 99.5% identical to the F protein of a mammalian MPV variant A1 as represented by the prototype NL/1/00 (SEQ ID NO:314); if the amino acid sequence of its M2-1 protein is at least 99% or at least 99.5% identical to the M2-1 protein of a mammalian MPV variant A1 as represented by the prototype NL/1/00 (SEQ ID NO:338); if the amino acid sequence of its M2-2 protein is at least 96% or at least 99% or at least 99.5% identical to the M2-2 protein of a mammalian MPV variant A1 as represented by the prototype NL/1/00 (SEQ ID NO:346); if the amino acid sequence of its SH protein is at least 84%, at least 90%, at least 95%, at least 98%, or at least 99% or at least 99.5% identical to the SH protein of a mammalian MPV variant A1 as represented by the prototype NL/1/00 (SEQ ID NO:382); and/or if the amino acid sequence of its L protein is at least 99% or at least 99.5% identical to the L protein of a virus of a mammalian MPV variant A1 as represented by the prototype NL/1/00 (SEQ ID NO:330).

An isolate of mammalian MPV is classified as a variant A2 if it is phylogenetically closer related to the viral isolate NL/17/00 (SEQ ID NO:20) than it is related to any of the following other viral isolates: NL/1/99 (SEQ ID NO:18), NL/1/00 (SEQ ID NO:19) and NL/1/94 (SEQ ID NO:21). One or more of the ORFs of a mammalian MPV can be used to classify the mammalian MPV into a variant. A mammalian MPV can be classified as an MPV variant A2, if the amino acid sequence of its G protein is at least 66%, at least 70%, at least 75%, at least 80%, at least 85%, at least 90%, at least 95%, at least 98%, at least 99% or at least 99.5% identical to the G protein of a mammalian MPV variant A2 as represented by the prototype N/17/00 (SEQ ID NO:332); if the amino acid sequence of its N protein is at least 99.5% identical to the N protein of a mammalian MPV variant A2 as represented by the prototype NL/17/00 (SEQ ID NO:367); if the amino acid sequence of its P protein is at least 96%, at least 98%, at least 99% or at least 99.5% identical to the P protein of a mammalian MPV variant A2 as represented by the prototype NL/17/00 (SEQ ID NO:375); if the amino acid sequence of its M protein is at least 99%, or at least 99.5% identical to the M protein of a mammalian MPV variant A2 as represented by the prototype NL/17/00 (SEQ ID NO:359); if the amino acid sequence of its F protein is at least 98%, at least 99% or at least 99.5% identical to the F protein of a mammalian MPV variant A2 as represented by the prototype NL/17/00 (SEQ ID NO:315); if the amino acid sequence of its M2-1 protein is at least 99%, or at least 99.5% identical to the M2-1 protein of a mammalian MPV variant A2 as represented by the prototype NL/17/00 (SEQ ID NO: 339); if the amino acid sequence of its M2-2 protein is at least 96%, at least 98%, at least 99% or at least 99.5% identical to the M2-2 protein of a mammalian MPV variant A2 as represented by the prototype NL/17/00 (SEQ ID NO:347); if the amino acid sequence of its SH protein is at least 84%, at least 85%, at least 90%, at least 95%, at least 98%, at least 99% or at least 99.5% identical to the SH protein of a mammalian MPV variant A2 as represented by the prototype NL/17/00 (SEQ ID NO:383); if the amino acid sequence of its L protein is at least 99% or at least 99.5% identical to the L protein of a mammalian MPV variant A2 as represented by the prototype NL/17/00 (SEQ ID NO:331).

An isolate of mammalian MPV is classified as a variant B2 if it is phylogenetically closer related to the viral isolate NL/1/94 (SEQ ID NO:21) than it is related to any of the following other viral isolates: NL/1/99 (SEQ ID NO:18), NL/1/00 (SEQ ID NO:19) and NL/17/00 (SEQ ID NO:20). One or more of the ORFs of a mammalian MPV can be used to classify the mammalian MPV into a variant. A mammalian MPV can be classified as an MPV variant B2, if the amino acid sequence of its G protein is at least 66%, at least 70%, at least 75%, at least 80%, at least 85%, at least 90%, at least 95%, at least 98%, or at least 99% or at least 99.5% identical to the G protein of a mammalian MPV variant B2 as represented by the prototype NL/1/94 (SEQ ID NO:325); if the amino acid sequence of its N protein is at least 99% or at least 99.5% identical to the N protein of a mammalian MNPV variant B2 as represented by the prototype NL/1/94 (SEQ ID NO:369); if the amino acid sequence of its P protein is at least 96%, at least 98%, or at least 99% or at least 99.5% identical to the P protein of a mammalian MPV variant B2 as represented by the prototype NL/1/94 (SEQ ID NO:377); if the amino acid sequence of its M protein is identical to the M protein of a mammalian MPV variant B2 as represented by the prototype NL/1/94 (SEQ ID NO:361); if the amino acid sequence of its F protein is at least 99% or at least 99.5% identical to the F protein of a mammalian MPV variant B2 as represented by the prototype NL/1/94 (SEQ ID NO:317); if the amino acid sequence of the M2-1 protein is at least 98% or at least 99% or at least 99.5% identical to the M2-1 protein of a mammalian MPV variant B2 as represented by the prototype NL/1/94 (SEQ ID NO:341); if the amino acid sequence that is at least 99% or at least 99.5% identical to the M2-2 protein of a mammalian MPV variant B2 as represented by the prototype NL/1/94 (SEQ ID NO:349); if the amino acid sequence of its SH protein is at least 84%, at least 85%, at least 90%, at least 95%, at least 98%, or at least 99% or at least 99.5% identical to the SH protein of a mammalian MPV variant B2 as represented by the prototype NL/1/94 (SEQ ID NO:385); and/or if the amino acid sequence of its. L protein is at least 99% or at least 99.5% identical to the L protein of a mammalian MPV variant B2 as represented by the prototype NL/1/94 (SEQ ID NO:333).

In certain embodiments, the percentage of sequence identity is based on an alignment of the full length proteins. In other embodiments, the percentage of sequence identity is based on an alignment of contiguous amino acid sequences of the proteins, wherein the amino acid sequences can be 25 amino acids, 50 amino acids, 75 amino acids, 100 amino acids, 125 amino acids, 150 amino acids, 175 amino acids, 200 amino acids, 225 amino acids, 250 amino acids, 275 amino acids, 300 amino acids, 325 amino acids, 350 amino acids, 375 amino acids, 400 amino acids, 425 amino acids, 450 amino acids, 475 amino acids, 500 amino acids, 750 amino acids, 1000 amino acids, 1250 amino acids, 1500 amino acids, 1750 amino acids, 2000 amino acids or 2250 amino acids in length.

The compositions/medicaments and treatments afforded according to the present invention take advantage of the unique abilities of antibodies, especially neutralizing antibodies, most especially high affinity, high specificity neutralizing antibodies such as those utilized herein, to control the ravages of bacterial and viral infections, most especially as they affect the delicate tissues of the respiratory system, and thereby offset the otherwise deleterious effects of relying solely on highly potent, and potentially toxic, antimicrobial agents that must, because of their chemical and biological properties, perforce be administered in sparing, and sometimes less than effective, dosages.

More specifically, the availability of compositions containing reduced amounts of such potent drugs along with accompanying antibodies, including high affinity antibodies, would serve to provide a middle ground for treatment and/or prevention of viral-induced diseases, such as those of the respiratory system, especially those caused by metapneumovirus (MPV), more in particular human metapneumovirus (hMPV).

The present invention is directed to therapeutically effective compositions comprising a neutralizing monoclonal antibody, including high affinity neutralizing antibodies, against respiratory viruses, such as metapneumovirus (MPV), more in particular human metapneumovirus (hMPV), as well as related viral agents causing respiratory disease, and other therapeutic agents, including other antibodies and nonantibody agents, useful in the treatment of respiratory disease.

It is thus an object of the present invention to provide therapeutic compositions comprising one or more neutralizing antibodies, including high affinity neutralizing antibodies, especially anti-MPV antibodies, as well as one or more additional agents capable of working either separately or in concert to treat and/or prevent antiviral infections, or otherwise combat and/or relieve the deleterious physiological and/or immunological effects of such infections, especially infections of the respiratory system, most especially diseases caused by metapneumovirus (MPV), more in particular human metapneumovirus (hMPV), and secondary infections associated therewith.

In accordance with the present invention, the neutralizing antibodies useful in the methods disclosed herein typically have affinity constants for their respective antigenic epitopes that are in the range of no greater than about 1 nM (or at least about 10-9 M). Because such high affinities are not easily measured, such value may commonly be considered as part of a range and may, for example, be within 2 fold of the nM values recited herein. Thus, they may be about 2 fold greater or lower than this value of may equal this value and still be useful in the present invention. Because this is a dissociation constant, the higher the value, the greater the degree of dissociation of the antigen and antibody and thus the lower the affinity. Such values may be easily converted to association constants by taking the reciprocal of the dissociation constant and adjusting the units to reciprocal molar in place of molar. In such case, the affinity of the antibody for its antigen will increase with increasing association constants.

With the advent of methods of molecular biology and recombinant technology, it is now possible to produce antibodies for use in the present invention by recombinant means and thereby generate gene sequences that code for specific amino acid sequences found in the polypeptide structure of the antibodies. This has permitted the ready production of antibodies having sequences characteristic of neutralizing antibodies from different species and sources.

The anti-MPV antibodies, including high affinity antibodies, useful in the compositions of the present invention will commonly comprise a mammalian, preferably a human, constant region and a variable region, said variable region comprising heavy and light chain framework regions and heavy and light chain CDRs, wherein the heavy and light chain framework regions are derived from a mammalian antibody, preferably a human antibody, and wherein the CDRs are derived from an antibody of some species other than a human, preferably a mouse. Where the framework amino acids are also derived from a non-human, the latter is preferably a mouse.

In addition, antibodies of the invention, including high affinity antibodies, bind the same epitope as the antibody from which the CDRs are derived, and wherein at least one of the CDRs of said antibody, including high affinity antibodies, contains amino acid substitutions, and wherein said substitutions comprise the replacement of one or more amino acids in the CDR regions by non-identical amino acids, preferably the amino acids of the correspondingly aligned positions of the CDR regions of the human antibody contributing the framework and constant domains. The contemplated host intended for treatment or prophylaxis with the compositions disclosed herein is generally an animal, especially a mammal, most especially a human patient.

Additional infectious agents acting as opportunistic pathogens are not limited to the viruses and bacteria. Thus, additional infection may be caused by non-viral or bacterial organisms, including various fungi and other parasites. As a result, the compositions according to the present invention may also comprise anti-infectious agents other than antiviral agents. Therapeutically active compositions within the present invention may thus comprise an anti-MPV antibody and an antibacterial agent, including antibiotics, as well as antifungal agents and antiparasitic agents of a broad or narrow spectrum. In addition, all of the latter additional agents may themselves be low or high affinity polyclonal or monoclonal antibodies with specificity against bacteria, or fungi, or other parasites infecting the respiratory system, as well as other related or unrelated systems.

The compositions disclosed according to the present invention for therapy of diseases as recited herein can easily include multiple antibodies against the same or different viruses, or against a virus and an addition microbial infectious agent, or against some non-viral microbial infectious agent, and may additionally include non-immunological agents in combination with said antibodies. In specific embodiments of the present invention, compositions disclosed herein may include an antibody against a virus, such as metapneumovirus (MPV), more in particular human metapneumovirus (hMPV), plus an antibody against a bacterial agent, especially one that infects the respiratory system, such as that causing tuberculosis, and, optionally, an antiviral agent. A therapeutic composition within the present invention may likewise comprise an antiviral antibody, a non-immunological antiviral agent, such as ribavirin, amantadine, rimantadine, or a neuraminidase-inhibitor, where MPV is the primary infectious agent, and an antimicrobial agent effective in the treatment of some non-viral pathogen, such as bacteria, including the agent for bacterial pneumonia, tuberculosis, or against some parasitic agent.

Thus, in accordance with a highly specific embodiment of the present invention, the anti-infectious agent used in combination with an anti-MPV antibody, including high affinity antibodies, may be an antibacterial agent, including but not limited to a macrolide, a penicillin, a cephalosporin, or a tetracycline, or may be an antifungal agent, including but not limited to amphotericin b, fluconazole, or ketoconazole, or an antiparasitic agent, including but not limited to trimethoprim, pentamidine, or a sulfonamide. The anti-infectious agent may be an antiviral agent such as ribavirin, amantadine, rimantadine, or a neuraminidase-inhibitor. Such additional agents can also include agents useful against other viruses as well as other agents useful against metapneumovirus (MPV), more in particular human metapneumovirus (hMPV).

However, in all highly preferred embodiments of the present invention the primary disease to be treated and/or prevented using the compositions disclosed herein is caused by metapneumovirus (MPV), more in particular human metapneumovirus (hMPV).

The invention thus provides the use of a nucleoside analog, preferably Ribavirin or a derivative thereof, and an antimicrobial neutralising antibody, preferably anti-hMPV, for the manufacture of a medicament for treating or preventing respiratory tract infections in a subject infected with a mammalian MPV such as hMPV. Also provided herein is the use of a nucleoside analog and an antimicrobial neutralising antibody for the manufacture of a medicament for treating or preventing respiratory tract infections in a subject infected with a mammalian MPV and co-infected with another virus, preferably one or more viruses from the Paramyxoviridae family, for example a virus that belongs to the Pneumovirinae subfamily such as Respiratory Syncitial Virus (RSV). It should be noted that the activity of Ribavirin is not restricted to viruses only. For example, it has been reported that certain micro-organisms (e.g. Pseudomonas spp.) are sensitive to Ribavirin. (Kruszewska et al., Acta Pol Pharm 2002, 59(6):436-9). In one embodiment of the invention, use of a nucleoside analog and an antimicrobial neutralising antibody is provided for the manufacture of a medicament for treating or preventing respiratory tract infections in a subject infected with mammalian MPV and co-infected with one or more other respiratory pathogens such as viruses, bacteria, yeast, fungi, mycoplasma and other parasites. Preferably said respiratory tract infections comprise viral lower respiratory tract infections. Such a medicament may be used to treat or prevent a respiratory tract infection in a human subject, for instance an infant of less than 5 years old, preferably less than 2 years old.

Also, elderly human subjects can be treated with a medicament comprising a nucleoside analog (preferably Ribavirin) of the invention. Furthermore, said nucleoside and said antimicrobial neutralising antibody can be used for the manufacture of a medicament or pharmaceutical composition to treat a subject that additionally suffers from a disease or condition other than a respiratory tract infection, such as cystic fibrosis, non-Hodgkin lymphoma, asthma, bone marrow transplantion or kidney transplantation. In a further embodiment, use of a nucleoside analog, preferably Ribavirin, and an antimicrobial neutralising antibody is provided for the manufacture of a medicament for treating or preventing a respiratory tract infection in a human subject suffering from SARS.

It has been shown that Ribavirin alone is not effective against hepatitis C virus infection in the long term. When Ribavirin is used in combination with the drug interferon, researchers have found that about twice as many people as those using Ribavirin alone show a long term clearance of detectable hepatitis C virus from the blood. Also, it was reported that high doses of Ribavirin to treat hepatitis C can be associated with several side effects, including leukopenia and haemolytic anemia. In order to reduce the occurrence or severity of side effects, co-administration of Ribavirin with interferon alpha-2B has been introduced for the treatment of hepatitis C (Reichard et al., Lancet 1991;337:1058). In one aspect of the invention, a nucleoside analog is used for the manufacture of a medicament for treating or preventing respiratory tract infections caused by mammalian MPV, wherein said medicament further comprises an antimicrobial antibody and a cytokine, preferably interferon, such as interferon alpha, beta or gamma. More preferred, a medicament or pharmaceutical composition of the invention comprises Ribavirin, an antimicrobial antibody, preferably anti-hMPV, and interferon alpha-2B. Said interferon may be pegylated interferon. Pegylated interferon is produced when chemical substances called polyethylene glycol (PEG) are attached to interferon. The PEG attachment to interferon helps the interferon to act in a number of ways. It shields the interferon from the body, so that it slows the rate at which the immune system attacks and breaks down the interferon. In addition, the PEG-interferon molecule is larger. This means it is able to stay in the circulation for longer as it is less likely to leak out into other tissues and it is also filtered and removed by the kidneys at a slower rate.

The invention thus provides the use of a nucleoside analog, preferably Ribavirin or a derivative thereof, and an antimicrobial neutralising antibody, preferably anti-hMPV, for the manufacture of a medicament for treating or preventing respiratory tract infections in a subject infected with a mammalian MPV such as hMPV.

The invention thus contemplates the use of one or more antibody. antimicrobial agents (i.e. preferably at least one that target processes essential for MPV viral replication and at least one that targets opportunistic “microbial” agents (e.g. bacterial, viral, fungal pathogens that infect the respiratory system)), and one or more nonantibody MPV antimicrobial agents (preferably at least one, that targets MPV and at least one that targets opportunistic “microbial” agents)(i.e. other nucleoside analogs, inhibitors, antibacterial/antifungal agents etc), including ones yet to be discovered, for the preparation of a medicament capable of working either separately or in concert to treat and/or prevent antiviral infections and associated secondary infections, especially those of the respiratory system, more especially diseases caused by MPV infection.

In a preferred embodiment, the invention provides a method for treating or preventing respiratory tract infections in a subject infected with a mammalian MPV, said method comprising administering a nucleoside analog and antimicrobial neutralising antibody to said subject.

The compositions of the present invention are not limited in their mode of administration to the patient. Thus, such administration can include parenteral as well as oral administration, and thus include intravenous, intramuscular, pulmonary and nasal administration. However, because of the nature of the diseases to be controlled and the types of chemical entities making up the present compositions, a preferred mode of administration is directly through the respiratory system. The antiviral agents contemplated for use in the compositions of the present invention are commonly administered through the respiratory system, often in the form of an aerosol. Thus, for purposes of administration, such compositions can be in the form of an aerosol or other type of spray, especially a fine particle aerosol, as defined below.

Pharmaceutical compositions will comprise sufficient active antibody and antiviral agents, so as to produce a therapeutically effective amount of the composition, i. e., an amount sufficient to reduce the amount of infecting virus, for example, metapneumovirus (MPV), more in particular human metapneumovirus (hMPV). The pharmaceutical compositions will also contain a pharmaceutically acceptable carrier, which includes all kinds of diluents and/or excipients, which include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. A thorough discussion of pharmaceutically acceptable excipients is available in Remington's Pharmaceutical Sciences (Mack Pub. Co., N.J. 1991).

The present invention is also directed to methods of treating and/or preventing a respiratory disease, especially diseases caused by metapneumovirus (MPV), more in particular human metapneumovirus (hMPV), and associated secondary infections, comprising administering to an animal, especially a human patient, at risk thereof, or afflicted therewith, of a therapeutically effective amount of a composition selected from the group consisting of the compositions disclosed herein.

Thus, the present invention provides a method for treating an animal, especially a human patient, suffering from a lower respiratory disease, such as metapneumovirus (MPV), and wherein said disease is caused by a viral agent or bacterial agent, including cases where said microbial agent is not the main cause of distress but merely serves to exacerbate an already existing condition, such as by causing clinical complications thereof, including instances of superinfection. The compositions of the present invention may be administered in the form of an aerosol spray of fine particles. The compositions of the present invention may be administered directly to the lower respiratory tract (for treating children) or to the upper respiratory tract (for treating adults) by intra-nasal spray. Such sprays must be formed of fine particles, which includes pharmacologically acceptable particles containing a therapeutically active amount of the compositions disclosed herein, and wherein such particles are no larger than about 10 pm in diameter, preferably no larger than about 5 pm in diameter and most preferably no larger than about 2; j. m in diameter. Optimum dosages for the anti-MPV antibodies making up the compositions of the present invention may be in the range of 5 to 20 mg/kg of body weight.

Typically, Ribavirin is administered to a subject using a small-particle aerosol generator. Also, an endotracheal tube can be used to administer a nucleoside analog to a subject. A typical treatment regimen is 1-500, more preferably 10-100, more preferably 10-30 and for example 20 mg/ml Ribavirin as the starting solution in the drug reservoir of a small-particle aerosol generator, with continuous aerosol administration for 12-18 hours per day for 3 to 7 days. Using a drug concentration of 20 mg/ml the average aerosol concentration for a 12 hour delivery period would be 190 micrograms/liter of air. However, the dosage regimen of the nucleoside analog and the concentration used may be varied according to clinical insights.

Furthermore, a method is provided for treating or preventing respiratory tract infections in a subject infected with a mammalian MPV and co-infected with one or more viruses from the Paramyxoviridae family, said method comprising administering a nucleoside analog, preferably Ribavirin or a derivative thereof, and an antimicrobial neutralising antibody, preferably an anti-MPV antibody to said subject. A subject may be co-infected with one or more viruses from the Paramyxoviridae family belongs to the Pneumovirinae sub-family, such as Respiratory Syncitial Virus (RSV).

In another aspect, the invention provides a method for treating or preventing respiratory tract infections in a subject infected with mammalian MPV and co-infected with one or more other respiratory pathogens (i.e. opportunistic pathogens) such as viruses, bacteria, yeast, fungi and mycoplasma, said method comprising administering a nucleoside analog and antimicrobial neutralising antibody, to said subject. In a preferred embodiment, said nucleoside analog is Ribavirin. Said subject may for instance be co-infected with one or more other RNA viruses, for example with a member of the Coronavirus family such as a severe acute respiratory syndrome (SARS)-related Coronavirus. Preferably said respiratory tract infections comprise viral lower respiratory tract infections.

A method of the invention is advantageously used to treat or prevent disease in a human subject, preferably a human subject considered at high risk for viral infections such as an infant subject of less than 5 years old, preferably less than 2 years old, an elderly subject, or an immunocompromised subject. In an immunocompromised subject, the immune system is functioning below normal. This makes them more susceptible to viral, fungal, or bacterial infections. Those who can be considered to be immunocompromised include AIDS patients (or HIV positive), diabetics, transplant patients (on immunosuppressive drugs), and those who are receiving chemotherapy for cancer.

In a further embodiment, said human subject additionally suffers from a disease or condition other than a respiratory tract infection, for example cystic fibrosis, non-Hodgkin lymphoma, asthma, bone marrow transplantation or kidney transplantation. A method of the invention can also be used to treat or prevent respiratory tract infections in a subject infected with a mammalian MPV wherein said subject suffers from SARS, said method comprising administering a (guanosine) nucleoside analog; preferably Ribavirin, and an antimicrobial neutralising antibody, preferably anti-hMPV to said subject.

Another preferred embodiment of the invention provides a method of treating upper and/or lower respiratory tract diseases in a host, especially that caused by metapneumovirus (MPV), more in particular human metapneumovirus (hMPV), and associated secondary viruses, susceptible to or suffering from such disease, comprising administering to the host a therapeutically effective amount of a composition comprising an antibody, preferably an anti-MPV antibody, an antiviral agent other than the previously stated antibody, with activity against MPV and an anti-inflammatory agent, said composition being sufficiently active as to produce a therapeutic effect against said disease or to protect against said disease. Such diseases include all manner of respiratory diseases, especially those caused by, or complicated by, MPV infections. Thus, the antimicrobial compositions of the present invention are also useful against other microbial agents besides MPV, especially where such other microbial agents, such as viruses or bacteria and the like, act as opportunistic agents to aggravate an already existing infection, such as an MPV infection, or where the presence of such non-MPV agent acts to make treatment of the respiratory infection more difficult. Of course, the clinical use of any composition of the present invention is a clinical decision to be made by the clinician and the exact course of such treatment is left to the clinician's sound discretion, with all such courses of treatment deemed within the bounds of the present invention.

Said composition may be administered by any available means, including but not limited to, oral, intravenous, intramuscular, pulmonary and nasal routes, and wherein said composition is present as a solution, a suspension or an aerosol spray, especially of fine particles. Such composition may be administered directly to the upper or lower respiratory tract of the host. The virus to be treated is metapneumovirus (MPV), more in particular human metapneumovirus (hMPV), but other viruses may be treated simultaneously, such as a member of the Paramyxoviridae family, a member of the Coronavirus family, more preferred a SARS-related Coronavirus, and other known and yet to be discovered viral respiratory “microbial” pathogens. In accordance with the methods of treatment disclosed herein, the non-antibody antiviral agent may be ribavirin, amantadine, rimantadine, or a neuraminidase-inhibitor. Such compositions can also include an immunoglobulin, such as human immunoglobulin G, which comprises antibodies against MPV or some other opportunistic virus.

### EXAMPLE

### Example 1

### Inhibitory Effect of Ribavirin on hMPV Replication

Materials and Methods

Cell Lines and Virus

Vero cells (African green monkey kidney cells) were maintained and passaged in Iscove's Modified Dulbecco's Medium (IMDM) containing L-glutamine (2 mM), penicillin (100 U/ml), streptomycin (100 μg/ml) and 10% fetal calf serum (FCS) and incubated in a humidified atmosphere at 37° C. Virus infection medium contained bovine serum albumin (BSA; 0.3%) and trypsin (2.5×10⁻⁴%) instead of FCS. Human Metapneumovirus stocks were prepared by inoculating Vero cells with hMPV, and harvesting 7-10 days post-infection, when there were visible cytopathic effects. The virus was stored at −70° C. and titrated by end point dilution on Vero cells.

Ribavirin as a Prophylactic

One day prior to infection, the cells were seeded in 24 well plates at 30-40% confluency, and incubated overnight at 37° C. The next day, cells were pre-incubated with fresh medium containing 0, 5, 25 or 50 μg/ml of Ribavirin for 2 hours prior to virus infection. Following this pre-incubation, cells were infected with 10 or 100 TCID50 (tissue culture infectious doses) of hMPV, in the continuing presence of Ribavirin. After 2 hours, the virus inoculum was washed off and fresh medium containing Ribavirin was put onto the cells.

Ribavirin as a Therapeutic

Cells were seeded in 24 well plates as described above. The next day, cells were inoculated with 10 or 100 TCID50 of hMPV in the absence of Ribavirin. Two hours post-infection, the supernatant was aspirated and the cells were washed in phosphate-buffered saline (PBS) to remove virus inoculum. Fresh medium was added and the cells were incubated at 37° C. At different time points (2, 4, 8 and 16 hours) after infection, Ribavirin was added to the cells to reach concentrations of 0, 5, 25 or 50 μg/ml.

Analysis of Ribavirin Effect

The plates were incubated at 37° C. for 1 or 3 days. Medium was refreshed every second day and Ribavirin was maintained in the medium at all times at the appropriate concentration. After the indicated incubation period, medium was removed, the cells were washed with PBS and cells were fixed with 80% acetone. Staining for immune fluorescence analysis was performed by incubating infected cells with guinea pig anti-hMPV serum in PBS for 1 hour, followed by a FITC-labeled rabbit anti-guinea pig polyclonal antibody preparation (DAKO) in PBS for 1 hour. Background staining was finally performed with eriochrome black before analysis under an immune fluorescence microscope. Infected cells were counted in 5 fields under high power magnification (320×). Results are presented as the total number of infected cells counted in these 5 fields

Results

Ribavirin as a Prophylactic

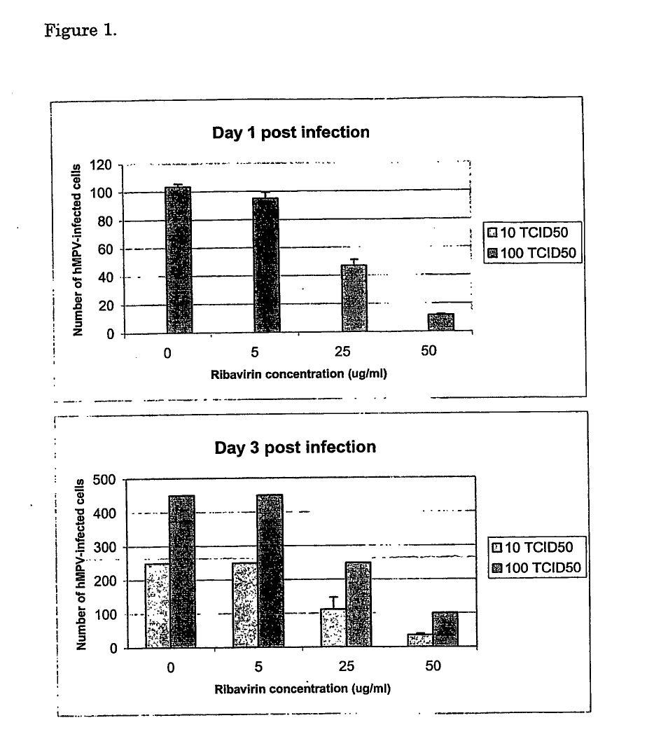

The results from the immune fluorescence analysis are presented in FIG. 1. At day 1 post-infection, few infected cells were visible in the wells infected with 10 TCID50 of hMPV. In contrast, in wells infected with 100 TCID50 of virus, there were sufficient infected cells to count. FIG. 1 demonstrates that the addition of Ribavirin shows a clear dose-dependent effect on virus replication, with a reduction in number of infected cells of approximately 50% in the presence of 25 μg/ml Ribavirin and of approximately 95% with 50 μg/ml of Ribavirin.

The same effect was seen at day 3 after infection for cells infected with both 10 or 100 TCID50 of virus. This is represented in FIG. 1 as 500 infected cells as each field under the microscope represents approximately 100 cells. The third time point was at day 6 after infection, at which time almost all cells were infected (data not shown).

Ribavirin as a Therapeutic

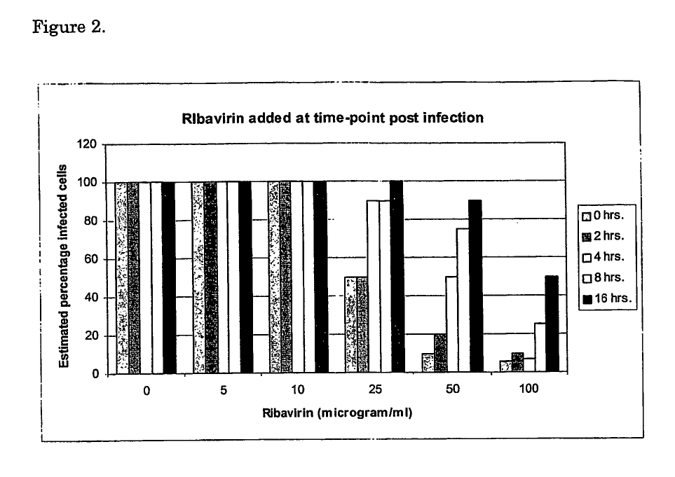

The results depicted in FIG. 2 demonstrate that Ribavirin inhibits replication of hMPV when added post-infection. Furthermore, the data indicate that the higher the concentration of Ribavirin and the earlier post-infection that Ribavirin is added, the more effective it is at inhibiting hMPV replication. For example, Ribavirin at 100 μg/ml is effective at reducing infection by 95% when added at 0, 2, or 4 hours, by 80% when added 8 hrs post-infection and by 50% when added 16 hrs post-infection. In contrast, administration of Ribavirin in a dose of 25 μg/ml inhibits viral replication by 50% when added at 0 or 2 hrs post-infection, by 10% when added 4 or 8 hrs post-infection and insignificantly when added after 16 hrs.

Conclusions

Our results show that Ribavirin inhibits hMPV growth in cell culture both when added prior to or after virus infection. A decrease in the number of infected cells of up to 95% was measured when 50 μg/ml of Ribavirin was added to cell cultures 2 hrs prior to or at the same time as virus infection. In order to see a significant effect 8 or 16 hrs post-infection, the concentration of Ribavirin has to be increased to 100 μg/ml.

Inhibition of hMPV by Ribavirin and Neutralizing Antibodies.

Passaging and seeding Vero cells (clone 118) was done in standard DMEM medium, supplemented with 10% FCS, L-glutamin and antibiotics (penicillin and streptomycin). Cells were seeded approximately 1:4 in 48 wells plates, so that cells would be ±75% confluent the next day. Infecting cells was done in IMDM medium with % BSA, Pen/Strep and L-glutamin. Prior to infection, two different concentrations of hMPV strain NL/01/00 (50 and 250 TCID50/200 μl were incubated for 1 hour at 37° C. with different dilutions of virus neutralizing guinea pig antiserum. Previous experiments had indicated that the antiserum we used could neutralize hMPV at a maximum dilution of 320×. We set this neutralizing limit at 1 VND and incubated the virus with 4, 1, ¼, 1/16, 1/64 and 0 VND (corresponding with a final serum dilution of 80×, 320×, 1280×, 5120×, 20480× and no serum).

Subsequently, cells were infected with either 50 or 250 TCID50 of hMPV (=200 μl of virus/antibody premix) and incubated with Ribavirin so that the final concentration of ribavirin was 0, 10, 25, 50 or 100 μg/ml. Plates were finally spun at 1500 g for 5 minutes and incubated at 37C in a humidified atmosphere for 3 days. After 3 days, from duplicate wells of each experimental condition the cells and supernatant were harvested and frozen at −70 C. Aliquots from these virus cultures were used for RNA isolation and subsequent quantification of viral genomic copy numbers by real-time RT-PCR on a TAQMAN® machine. The other duplicate wells from each experimental condition were stained for hMPV by immunofluorescence and numbers of infected cells were counted under a fluorescence microscope. Briefly, cells were washed with PBS and fixed with 80% acetone, washed again and subsequently incubated with a guinea pig anti-hMPV serum. After removal of the antiserum, cells were incubated with a FITC-labeled rabbit-anti-guinea pig polyclonal antibody preparation, washed and analyzed.

Results

Note: Indicated results are estimated percentages of infected cells. Viral genomic copy numbers remain to be determined by taqman analyses (to be done this week)

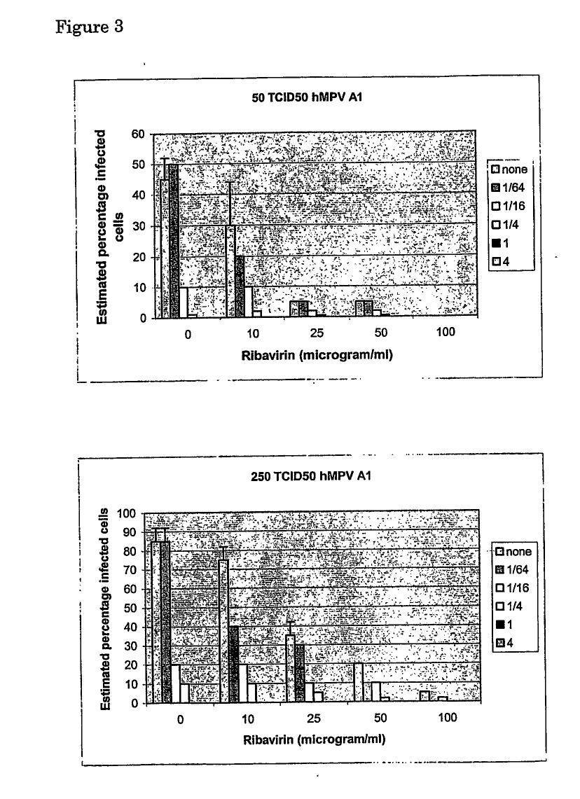

As expected, both ribavirin and antiserum have a profound effect on the growth of hMPV in vero cells in vitro (FIG. 3) At a concentration of 25 μg/ml, ribavirin inhibits viral proliferation by about 90%. A similar inhibition of viral replication can be reached by incubation with antiserum at 1/4 of the virus neutralizing dose (VND).

1-12. (canceled)

13. A method for treating or preventing respiratory tract infections in a subject infected with a mammalian Metapneumovirus (MPV), said method comprising administering a nucleoside analog and an antimicrobial neutralizing antibody to said subject.

14. The method of claim 13, wherein said subject is co-infected with one or more viruses from the Paramyxoviridae family.

15. The method of claim 14, wherein said subject is co-infected with a virus that belongs to the Pneumovirinae sub-family.

16. A method for treating or preventing respiratory tract infections in a subject infected with mammalian MPV and co-infected with one or more other respiratory pathogens, said method comprising administering a nucleoside analog and an antimicrobial neutralizing antibody to said subject.

17. The method of claim 16, wherein said subject is co-infected with one or more other RNA viruses.

18. The method of claim 13, wherein said nucleoside analog comprises ribavirin or a derivative thereof.

19. The method of claim 13, wherein said mammalian MPV is hMPV.

20. The method of claim 13, wherein said antimicrobial neutralizing antibody comprises an anti-hMPV antibody.

21. The method of claim 13, wherein said respiratory tract infections comprise viral lower respiratory tract infections.

22. The method of claim 13, wherein the subject is human.

23. The method of claim 22, wherein the subject is less than 5 years old, or wherein the subject is elderly.

24. (canceled)

25. The method of claim 22, wherein the subject additionally suffers from a disease or condition other than a respiratory tract infection.

26. The method of claim 22, wherein the subject is immunocompromised.

27. The method of claim 22, wherein the subject suffers from severe acute respiratory syndrome (SARS).

28. The method of claim 25, wherein said disease or condition is cystic fibrosis, non-Hodgkin lymphoma, asthma, bone marrow transplantation or kidney transplantation.

29. The method of claim 16, wherein said nucleoside analog comprises ribavirin or a derivative thereof.

30. The method of claim 16, wherein said mammalian MPV is hMPV.

31. The method of claim 16, wherein said antimicrobial neutralizing antibody comprises an anti-hMPV antibody.

32. The method of claim 16, wherein said respiratory tract infections comprise viral lower respiratory tract infections.

33. The method of claim 16, wherein the subject is human.

34. The method of claim 33, wherein the subject is less than 5 years old or wherein the subject is elderly.

35. The method of claim 33, wherein the subject additionally suffers from. a disease or condition other than a respiratory tract infection.

36. The method of claim 33, wherein the subject is immunocompromised.

37. The method of claim 33, wherein the subject suffers from severe acute respiratory syndrome (SARS).

38. The method of claim 35, wherein said disease or condition is cystic fibrosis, non-Hodgkin lymphoma, asthma, bone marrow transplantation or kidney transplantation.


SEQUENCE LISTING (33 sequences)

SEQ ID NO: 1 - protein, 15 aa, Artificial
  linker
GGGGSGGGGSGGGGS

SEQ ID NO: 2 - protein, 15 aa, Artificial
  linker
ESGRSGGGGSGGGGS

SEQ ID NO: 3 - protein, 14 aa, Artificial
  linker
EGKSSGSGSESKST

SEQ ID NO: 4 - protein, 15 aa, Artificial
  linker
EGKSSGSGSESKSTQ

SEQ ID NO: 5 - protein, 14 aa, Artificial
  linker
EGKSSGSGSESKVD

SEQ ID NO: 6 - protein, 14 aa, Artificial
  linker
GSTSGSGKSSEGKG

SEQ ID NO: 7 - protein, 18 aa, Artificial
  linker
KESGSVSSEQLAQFRSLD

SEQ ID NO: 8 - protein, 16 aa, Artificial
  linker
ESGSVSSEELAFRSLD

SEQ ID NO: 9 - protein, 4 aa, Artificial
  endoplasmatic reticulum localization signal
KDEL

SEQ ID NO: 10 - protein, 4 aa, Artificial
  endoplasmatic reticulum localization signal
DDEL

SEQ ID NO: 11 - protein, 4 aa, Artificial
  endoplasmatic reticulum localization signal
DEEL

SEQ ID NO: 12 - protein, 4 aa, Artificial
  endoplasmatic reticulum localization signal
QEDL

SEQ ID NO: 13 - protein, 4 aa, Artificial
  endoplasmatic reticulum localization signal
RDEL

SEQ ID NO: 14 - protein, 7 aa, Artificial
  nucleus localization sequence
PKKKRKV

SEQ ID NO: 15 - protein, 7 aa, Artificial
  nucleus localization sequence
PQKKIKS

SEQ ID NO: 16 - protein, 5 aa, Artificial
  nucleus localization sequence
QPKKP

SEQ ID NO: 17 - protein, 4 aa, Artificial
  nucleus localization sequence
RKKR

SEQ ID NO: 18 - protein, 12 aa, Artificial
  nucleolar region localization sequence
RKKRRQRRRAHQ

SEQ ID NO: 19 - protein, 16 aa, Artificial
  nucleolar region localization sequence
RQARRNRRRRWRERQR

SEQ ID NO: 20 - protein, 19 aa, Artificial
  nucleolar region localization sequence
MPLTRRRPAASQALAPPTP

SEQ ID NO: 21 - protein, 15 aa, Artificial
  endosomal compartment localization signal
MDDQRDLISNNEQLP

SEQ ID NO: 22 - protein, 32 aa, Artificial
  mitochondrial matrix localization signal
  MISC_FEATURE (7)..(8): Xaa can be any amino acid
  MISC_FEATURE (32)..(32): Xaa can be any amino acid
MLFNLRXXLNNAAFRHGHNFMVRNFRCGQPLX

SEQ ID NO: 23 - protein, 8 aa, Artificial
  plasma membrane localization signal
GCVCSSNP

SEQ ID NO: 24 - protein, 8 aa, Artificial
  plasma membrane localization signal
GQTVTTPL

SEQ ID NO: 25 - protein, 8 aa, Artificial
  plasma membrane localization signal
GQELSQHE

SEQ ID NO: 26 - protein, 8 aa, Artificial
  plasma membrane localization signal
GNSPSYNP

SEQ ID NO: 27 - protein, 8 aa, Artificial
  plasma membrane localization signal
GVSGSKGQ

SEQ ID NO: 28 - protein, 8 aa, Artificial
  plasma membrane localization signal
GQTITTPL

SEQ ID NO: 29 - protein, 8 aa, Artificial
  plasma membrane localization signal
GQTLTTPL

SEQ ID NO: 30 - protein, 8 aa, Artificial
  plasma membrane localization signal
GQIFSRSA

SEQ ID NO: 31 - protein, 8 aa, Artificial
  plasma membrane localization signal
GQIHGLSP

SEQ ID NO: 32 - protein, 8 aa, Artificial
  plasma membrane localization signal
GARASVLS

SEQ ID NO: 33 - protein, 8 aa, Artificial
  plasma membrane localization signal
GCTLSAEE